(19) United States
(12) Patent Application Publication
(10) Pub. No.: US 2022/0362343 A1
(43) Pub. Date: Nov. 17, 2022

(54) METHOD OF REDUCING TET2 MUTANT ALLELE BURDEN

(71) Applicant: PharmaEssentia Corporation, Taipei (TW)

(72) Inventors: Ko-Chung Lin, Lexington, MA (US); Robert Kralovics, Vienna (AT)

(21) Appl. No.: 17/744,139

(22) Filed: May 13, 2022

Related U.S. Application Data
(60) Provisional application No. 63/188,706, filed on May 14, 2021.

(51) Int. Cl. A61K 38/21; A61K 47/60; A61P 7/00

(52) U.S. Cl. CPC A61K 38/212; A61K 47/60; A61P 7/00

(57) ABSTRACT

A method of reducing TET2 mutant allele burden in a subject, comprising: administering to a subject identified as having a TET2 mutation a 50 to 540 μg dose of a pegylated interferon-α at a regular interval of 2 to 8 weeks for a treatment period, the pegylated interferon-α being a conjugate of formula I:

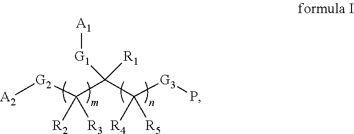

in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10,
   wherein the subject has a reduction in TET2 mutant allele burden at a second time point in the treatment period as compared to a first time point before or earlier in the treatment period.

### RELATED APPLICATION

This application claims priority to U.S. Provisional Application No. 63/188,706, filed on May 14, 2021, the entire content of which is incorporated by reference herein.

### BACKGROUND

Interferon-α is a causative treatment in polycythemia vera. Its targeted action on JAK2V617F mutated hematopoietic stem cells diminishes the tumor burden. However, up to 50% of patients with polycythemia vera present with additional non-driver mutations that promote disease progression, and which could affect responses to interferon-α.

The Ten-Eleven Translocation-2 (TET2) gene is an epigenetic regulator. Loss-of-function mutations of TET2 are found in a variety of blood cancers in both myeloid and lymphoid lineages. Clonal blood cells carrying leukemia-driver mutations have been detected in significant proportions of cohorts without blood cancers, those in the TET2 gene being second/third most frequent. The presence of the clonal blood cells predicts elevated risk for developing various blood cancers. TET2 inactivation through loss-of-function mutation, deletion or IDH½ (Isocitrate Dehydrogenase 1 and 2) gene mutation is a common event in myeloid and lymphoid malignancies. Increased TET2 mutation levels correlate with disease progression and transformation.

### SUMMARY

In one aspect, described herein is a method of reducing TET2 mutant allele burden in a subject. The method includes administering to a subject identified as having a TET2 mutation a 50 to 540 μg dose of a pegylated interferon-α at a regular interval of 2 to 8 weeks (e.g., 2 to 4 weeks) for a treatment period, the pegylated interferon-α being a conjugate of formula I:

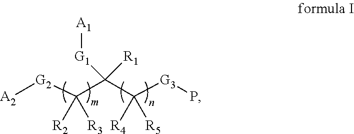

in which

each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;

each of A₁ and A₂, independently, is a polymer moiety;

each of G₁, G₂, and G₃, independently, is a bond or a linking functional group; P is an interferon-α moiety;

m is 0 or an integer of 1-10; and

n is an integer of 1-10,

wherein the subject has a reduction in TET2 mutant allele burden at a second time point in the treatment period as compared to a first time point before or earlier in the treatment period.

In some embodiments, the method further includes assaying and detecting the TET2 mutant allele burden in the subject at the first time point and the second time point. In some embodiments, the method includes assaying and detecting the TET2 mutant allele before the administering step. In some embodiments, the second time point is 3 years or less (e.g., 2 years or less, or 1 year or less).

In some embodiments, the reduction in TET2 mutant allele burden is maintained or increased beyond the second time point. In some embodiments, the TET2 mutant allele burden is reduced by at least 10% (e.g., at least 20% to 50%).

In some embodiments, the subject has essential thrombocythemia, polycythemia vera or myelofibrosis.

In some embodiments, the subject has a complete hematologic response at the second time point.

In some embodiments, the subject has a reduced JAK2V617F allele burden at the second time point as compared to the first time point.

In one aspect, described herein is a method of treating polycythemia vera in a subject, comprising: administering to a subject identified as having polycythemia vera and a TET2 mutation a 50 to 540 μg dose of a pegylated interferon-α at a regular interval of 2-8 weeks for a treatment period, the pegylated interferon-α being a conjugate of formula I:

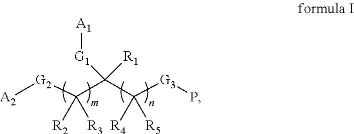

in which

each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;

each of A₁ and A₂, independently, is a polymer moiety;

each of G₁, G₂, and G₃, independently, is a bond or a linking functional group; P is an interferon-ca moiety;

m is 0 or an integer of 1-10; and

n is an integer of 1-10,

wherein the subject has a reduction in JAK2V617F allele burden at a second time point in the treatment period as compared to a first time point before or earlier in the treatment period, and optionally, a complete hematological response also at the second time point.

In some embodiments, the method further includes assaying and detecting the TET2 mutant allele burden in the subject at the first time point and the second time point. In some embodiments, the method includes assaying and detecting the TET2 mutant allele before the administering step. In some embodiments, the second time point is 3 years or less (e.g., 2 years or less, or 1 year or less).

In some embodiments, the reduction in TET2 mutant allele burden is maintained or increased beyond the second time point. In some embodiments, the TET2 mutant allele burden is reduced by at least 10% (e.g., at least 20% to 50%).

In some embodiments, the reduction in JAK2V617F allele burden is maintained or increased beyond the second time point. In some embodiments, the JAK2V617F mutant allele burden is reduced by at least 20%. In some embodiments, the complete hematological response is maintained beyond the second time point.

In some embodiments, the reduction in JAK2V617F allele burden or another response is not significantly different from a control polycythemia vera subject with wild-type TET2.

In any of the methods described herein, the conjugate can have one or more properties including: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate has one or more features including: G3 is a bond and P is an interferon-α moiety in which the amino group at the N-terminus is attached to G3; A₁ and A₂ are polyalkylene oxide moieties having a molecular weight of 10-30 kD; each of G₁ and G₂ is

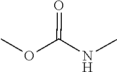

in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues. In some embodiments, the interferon-α moiety is a human interferon-α_{2b} having an extra proline residue at the N-terminus and is 166 amino acids in length.

In some embodiments, the conjugate is

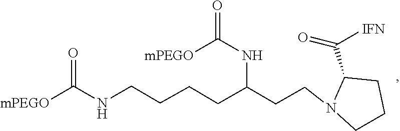

in which mPEG has a molecular weight of 20 kD and IFN is an interferon-α_{2b}.

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a graph showing mean dose of ropeginterferon alpha-2b administered to patients with wildtype or mutant TET2.

FIG. 2 is a graph showing complete hematological response among patients with wildtype or mutant TET2.

### DETAILED DESCRIPTION

It was unexpectedly discovered that a pegylated interferon can reduce the TET2 mutant allele burden in a subject. Further, it was found that, unexpectedly, having a TET2 mutation does not significantly impact a polycythemia vera patient's response to treatment with a pegylated interferon.

Described herein is a method of reducing TET2 mutant allele burden in a subject using a pegylated interferon-α. Also described herein is a method of treating a subject having polycythemia vera and a TET2 mutation using a pegylated interferon-α.

TET2 mutations are found in peripheral blood cells in older adults, and in patients with myelodysplastic syndrome, myelodysplastic syndrome/myeloproliferative neoplasms, myeloproliferative neoplasms, or acute myeloid leukemia (AML). For example, TET2 mutations occur in patients with myelofibrosis, polycythemia vera, or essential thrombocythemia. The mutations lead to clonal hematopoiesis of indeterminate potential (CHIP) with skewed myelomonocytic differentiation. CHIP is associated with an increased risk of transformation to a hematological malignancy.

TET2 mutations can include frame shifts, generated stop codons, in-frame deletions, and amino acid substitutions. See, e.g., Weissmann et al., Leukemia, 26, 934-942 (2012); Chou et al., Blood, 118(14): 3803-3810 (2011); Schaub et al., Blood, 115(10): 2003-2007 (2010); and Ha et al., Annals of Clinical & Laboratory Science, 44(2): 173-179 (2014). Examples of TET2 mutations include: Thr1372Ile, Ser1285del, Gln891X, Leu1210Pro, Arg544X, splice donor c.3980+2G>T, Asp1858fs, Gly1275Glu, 1295A>G(Glu432Gly), 1285G>A(Gly429Arg), 744C>A(His248Gln), 115C>T(Pro39Ser), 803C>G(Ser268Trp), 3686T>G(Leu1229Arg), 3743T>C(Leu1248Pro), 3640C>T(Arg1214Trp), 3781C>T(Arg1261Cys), 3662G>A(Cys1221Tyr), 3621G>T(Glu1207Asp), 812G>C(Cys1271Ser), 3812G>A(Cys1271Tyr), 3866G>A(Cys1289Tyr), 3818G>C(Cys1273Ser), 3866G>T(Cys1289Phe), 3860T>C(Phe1287Ser), 4019T>G(Leu1340Arg), 3980A>C(Gln1327Pro), 3965T>A(Leu1322Gln), 4138C>T(His1380Tyr), 4064C>T(Ala1355Val), 4133G>A(Cys1378Tyr), 4513G>A(Ala1505Thr), 4188C>G(Cys1396Trp), 4256C>G(Pro1419Arg), 5608T>C(Ser1870Pro), 5885C>T(Pro1962Leu), V4664A>T(Glu1555Val), 5711A>G(His1904Arg), 5735 A>C(His1912Pro), 5618T>C(Ile1873Thr), and 5666C>G(Pro1889Arg). A subject can have more than one TET2 mutations.

TET2 mutations can be assayed in blood samples or blood cells using methods known in the art, e.g., sequencing or PCR. TET2 mutant allele burden (%) can also be determined using methods known in the art. Allele burden represents the percentage of mutant TET2 alleles present among all TET2 alleles in blood cells, e.g., peripheral blood mononuclear cells. More specifically, a TET2 allele burden can be at least 5%, e.g., about 5%, 10%, 15%, 20%, 25%, is 30%, 35%, 40%, 45%, 50%, 55%, 60% or more.

Administering a pegylated interferon described herein can reduce the TET2 mutant allele burden in a subject. A reduction of the TET2 mutant allele burden can be at least 5% (e.g., 5%, 10%, 15%, 20%, 25%, 30%, 35%, 40%, 45%, 50%, 55%, 60% or more) over a defined period. The allele burden can decline to 50% or less, e.g., 50% or less, 45% or less, 40% or less, 37% or less, 35% or less, 30% or less, 25% or less, 20% or less, 15% or less, 10% or less, 7% or less, 5% or less, or 1% or less. The defined period can be a treatment period or the period between two time points. The period between two time points can be three years or less (e.g., 6 months or less, 12 months or less, 18 months or less, 24 months or less, or 30 months or less). The earlier time point can occur before or during a treatment period. The reduction in TET2 mutant allele burden can be maintained or increased over a period, e.g., a longer treatment period or for a period beyond the defined period.

A treated subject exhibiting a reduced TET2 mutant allele burden can also have a reduced JAK2617F allele burden, a hematological response (partial or complete), or other indicators of a good response to the treatment.

As compared to a subject treated with HU or another interferon, a subject treated with a pegylated interferon described herein can exhibit a greater reduction in TET2 mutant allele burden. A subject with a TET2 mutant can respond better, as determined by art-accepted criteria, to treatment with a pegylated interferon described herein than a subject treated with HU or another interferon.

In any of the methods described herein, the presence or absence of a TET2 mutation or mutant allele burden in a subject can be determined at one or two more time points before, at the beginning, during, at the end, or after a treatment period.

A pegylated interferon-α used in any of the methods described herein can be a conjugate of formula I:

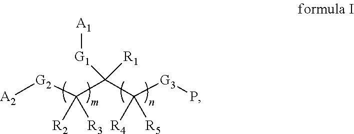

wherein each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl; each of A₁ and A₂, independently, is a polymer moiety; each of G₁, G₂, and G₃, independently, is a bond or a linking functional group; P is an interferon-α moiety; m is 0 or an integer of 1-10; and n is an integer of 1-10. The conjugate of formula I is also described in WO2009/023826A1.

Referring to the above formula, the conjugate can have one or more of the following features: G3 is a bond and P is an interferon-α moiety (e.g., a human interferon-α-2b) in which the amino group at the N-terminus is attached to G3; A₁ and A₂ are polyalkylene oxide moieties having a molecular weight of 2-100 kD (preferably 10-30 kD), each of G₁ and G₂ is

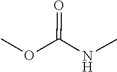

(in which O is attached to A₁ or A₂, and NH is attached to a carbon atom as shown in formula I), or each of G₁ and G₂ is urea, sulfonamide, or amide, (in which N is attached to a carbon atom as shown in formula I); m is 4, n is 2, and each of R₁, R₂, R₃, R₄, and R₅ is H; and the interferon-α moiety is a modified interferon-α moiety containing 1-4 additional amino acid residues. In some embodiments, the interferon-α moiety is a human interferon α-2b having an extra proline residue at the N-terminus and is 166 amino acids in length.

The conjugate can also have one or more of the following properties: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate is ropeginterferon alpha-2b, which has a predominant isoform having the formula:

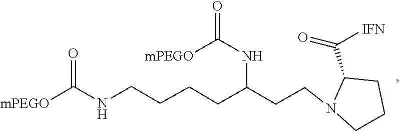

in which mPEG has a molecular weight of 20 kD and IFN is an interferon-α-2b (e.g., a human interferon-α-2b).

Ropeginterferon alpha-2b is produced by covalent attachment of a 40 kDa PEG molecule to the N-terminal proline residue of a Proline-Interferon-2b (Pro-IFN alpha-2b). Proline-interferon alpha-2b is generated by recombinant DNA technology introducing an extra proline residue to a human interferon α-2b at the N-terminus, giving a polypeptide of total 166 amino acids in length. Pro-IFN alpha-2b has a molecular weight of approximately 19 kDa and has the amino acid sequence identical to the theoretical sequence predicted excluding the additional N-terminal proline. It is then PEGylated with an approximately 40 kDa PEG moiety forming approximately 60 kDa PEGylated proline-interferon alpha-2b or ropeginterferon alpha-2b. The biological activity of ropeginterferon alpha-2b is determined by cytopathic effect (CPE)-based antiviral assay.

In any of the methods described herein, the pegylated interferon-α can be administered by any means known in the art, e.g., via subcutaneous or intravenous route. The pegylated interferon-α can be formulated as an injectable formulation. For example, it can be in the form of a ready-to-use prefilled syringe (PFS) containing, e.g., 0.2 to 2 mL of solution, that can be for self-injection. Each PFS can contain the labeled amount of the drug product, sodium chloride, sodium acetate anhydrous, acetic acid, benzyl alcohol, and polysorbate 80. The vehicle for the drug product can be sterile water for injection, and the drug product solution can have a pH of about 6.0.

The term “dose” refers to the amount of a compound administered to a subject at one time.

The term “interval” refers to the time between administration of two consecutive doses. In any of the methods described herein, the pegylated interferon-α is administered at an interval of 2 to 8 weeks, e.g., 2, 3, 4, 5, 6, 7, or 8 weeks. For example, a dose can be administered once every 2, 3, 4, 5, 6, 7, or 8 weeks. An interval that is defined in days or months is also contemplated. A regular interval of 10 to 60 days (e.g., 14, 21, 25, 26, 27, 28, 29, 30, 31, 35, 42, 49, and 56 days), one month, or two months can be utilized in any of the methods.

A treatment period can be at least 1, 2, 3, 4, 5, 6, 7, 8, 9, 10, 11, 12, 24, 36, 42, 48, 54, 60, 66, 72, 78, 84 or more months. In some embodiments, the treatment period is 1, 2, 3, 4, 4.5, 5, is 5.5, 6, 6.5, 7, 7.5, 8, 8.5, 9, 9.5, 10 or more years.

A dose administered during a treatment period ranges from 50 to 650 μg. The dose can be 50 μg, up to 55 μg, specifically up to 60 μg, specifically up to 65 μg, specifically up to 75 μg, specifically up to 80 μg, specifically up to 85 μg, specifically up to 90 μg, specifically up to 95 μg, specifically up to 100 μg, specifically up to 105 μg, specifically up to 110 μg, specifically up to 115 μg, specifically up to 120 μg, specifically up to 125 μg, specifically up to 130 μg, specifically up to 135 μg, specifically up to 140 μg, specifically up to 145 μg, specifically up to 150 μg, specifically up to 155 μg, specifically up to 160 μg, specifically up to 165 μg, specifically up to 170 μg, specifically up to 175 μg, specifically up to 180 μg, specifically up to 185 μg, specifically up to 190 μg, specifically up to 195 μg, specifically up to 200 μg, specifically up to 205 μg, specifically up to 210 μg, specifically up to 215 μg, specifically up to 225 μg, specifically up to 230 μg, specifically up to 235 μg, specifically up to 240 μg, specifically up to 245 μg, specifically up to 250 μg, specifically up to 255 μg, specifically up to 260 μg, specifically up to 265 μg, specifically up to 270 μg, specifically up to 275 μg, specifically up to 280 μg, specifically up to 285 μg, specifically up to 290 μg, specifically up to 295 μg, specifically up to 300 μg, specifically up to 305 μg, specifically up to 310 μg, specifically up to 315 μg, specifically up to 320 μg, specifically up to 325 μg, specifically up to 330 μg, specifically up to 335 μg, specifically up to 340 μg, specifically up to 345 μg, specifically up to 350 μg, specifically up to 400 μg, specifically up to 450 μg, specifically up to 500 μg, specifically up to 540 μg, or specifically up to 650 μg.

During any treatment period, the pegylated interferon-α can be administered at a constant dose, meaning that the same dose is administered each time or only minimally different doses are administered (e.g., dose variation or deviation of less than 10%, specifically less than 5%, specifically less than 1%). Alternatively, different doses can be administered at a regular interval during a treatment period. For example, the interferon can be administered at a particular dose at a regular interval for a certain time, and it can then be administered at a different dose (higher or lower than the first dose) at the same regular interval.

The subject can be a subject who has not been treated with an interferon before or a subject who had previously been administered a dose (e.g., 12.5, 15, 18.75, or 25 μg) of a type I interferon once per week or every two weeks. The subject can be a subject who has been treated previously with a therapy other than an interferon (e.g., HU).

A subject in need thereof can be treated with the pegylated interferon-α described herein using one dosage regimen for a time period and then switched to a different dosage regimen.

More specifically, a 50 to 650 μg dose of the pegylated interferon-α can be administered to a subject in need thereof at a first regular interval for a first treatment period, the first interval being 2 to 4 weeks (e.g., 2, 3, or 4 weeks), and subsequently, a 50 to 650 μg dose of the pegylated interferon-α is administered to the subject at a second regular interval for a second treatment period, the second interval being at least 3 weeks (e.g., 3, 4, 5, 6, 7, or 8 weeks).

Subjects who show a good response to a pegylated interferon-α dosage regimen can be switched to another regimen in which the interferon is administered at a lower dose, higher dose, and/or at a longer interval.

In some embodiments, the total amount of the pegylated interferon-α administered to the subject per a given period (e.g., 1 week, 2 weeks, 4 weeks, 1 month, or 2 months) during the second treatment period is lower or higher (e.g., lower or higher by 20%, 30%, 40%, 45%, 50%, or more) than the total amount administered per the same given period during the first treatment period. For example, the monthly total amount of the interferon administered during the second treatment period can be lower or higher (e.g., by 20%, 30%, 40%, 45%, 50%, or more) than the monthly total amount administered during the first treatment period.

The dose administered during the first treatment period and the dose administered during the second treatment period can be the same but at different intervals. Alternatively, the dose administered during the second treatment period can be lower or higher than the dose administered during the first treatment period.

The first treatment period and the second treatment period can separately be at least 1, 2, 3, 4, 5, 6, 7, 8, 9, 10, 11, 12, 24, 36, 42, 48, 54, 60, 66, 72, 78, 84 or more months. In some embodiments, each treatment period is, individually, at least 1, 2, 3, 4, 4.5, 5, 5.5, 6, 6.5, 7, 7.5, 8, 8.5, 9, 9.5, 10 or more years.

The first treatment period can continue until the subject shows a good response to the treatment. Whether a subject is responding well to the treatment can be determined by a practitioner skilled in the art based on art-accepted criteria (e.g., hematological parameter, allelic burden, symptoms, and adverse events).

In any of the methods or treatment periods described herein, the pegylated interferon-α may be titrated. A subject can be treated with a lower starting dose (e.g., 50 to 350 μg) of a pegylated interferon-α every 2 to 8 weeks. If the subject responds well (e.g., lack of significant drug-related adverse events, significant self-reported discomfort, abnormal hematological responses or other symptoms) after a time, the dose given to the subject may be increased incrementally (e.g., by 50 to 150 μg, 50 μg, 75 μg, 100 μg, 125 μg, or 150 μg, or a combination thereof) every 2 to 12 weeks (e.g., every 2, 3, 4, 5, 6, 7, 8, 9, 10, 11, or 12 weeks, or a combination thereof) until the dose reaches a target dose (e.g., at least 400 μg, 425 μg, 450 μg, 475 μg, 500 μg, 525 μg, 550 μg, or 650 μg). After that, the target dose is maintained during the treatment period. The dose can be increased successively until the desired dose is reached. For example, if the pegylated interferon-α is administered once every 2, 3, 4, 5, 6, 7, or 8 weeks, the dose can be increased every 2, 3, 4, 5, 6, 7 or 8 weeks, respectively. For example, a subject may be given a starting dose of 100 μg. If the subject responds well to the initial dose, the dose may be increased by 100 μg every two weeks until it reaches a target dose of 500 μg. In some embodiments, the target dose is reached in 4 to 48 weeks from the first administration of the pegylated interferon-α (e.g., 4 to 12 weeks, 4 to 16 weeks, 4 to 20 weeks, 4 to 24 weeks, 6 to 12 weeks, 6 to 16 weeks, 6 to 20 weeks, 6 to 24 weeks, 6 to 28 weeks, 6 to 32 weeks, 6 to 36 weeks, 6 to 40 weeks, 8 to 12 weeks, 8 to 16 weeks, 8 to 20 weeks, 8 to 24 weeks, 8 to 28 weeks, 8 to 32 weeks, 8 to 36 weeks, 8 to 40 weeks, 12 to 16 weeks, 12 to 20 weeks, 12 to 24 weeks, 12 to 28 weeks, 12 to 32 weeks, 12 to 36 weeks, 12 to 40 weeks, or 12 to 48 weeks). In some embodiments, the target dose is reached in 4 to 8 weeks from the initial administration of the pegylated interferon-α. During the titration process, any dose, prior to reaching the target dose, may be maintained for a time period (e.g., 4 to 16 weeks) or a number of successive doses (e.g., 2 to 8 successive doses) or reduced depending on the subject's response. In some embodiments, the target dose is reached within 2 to 4 successive doses.

An initial dose or starting dose of the pegylated interferon-α refers to the first dose administered to a subject during a treatment period (i.e., week 0), wherein, prior to the treatment period, the subject is interferon-treatment naïve or has not been administered the same pegylated interferon-α. A subject who is interferon-treatment naïve is a subject who has not been treated with any form of interferon, whether pegylated or non-pegylated (e.g., recombinant interferon, or peginterferon alpha-2b or peginterferon alpha-2a approved to be administered weekly).

Response criteria for assessing treatment can include symptoms and signs of the disease, peripheral blood counts (e.g., platelet counts and white blood cell counts), vascular events, signs of progression of disease, bone marrow histology, and molecular response. Response criteria can be defined based on consensus criteria in the art, e.g., the European LeukemiaNet (ELN) and International Working Group (IWG) criteria.

For example, any combination of the following criteria can be used to define a response for essential thrombocythemia or polycythemia vera: resolution of disease-related signs including palpable hepatosplenomegaly; large symptoms improvement; platelet count ≤400×10⁹/L; WBC count ≤10×10⁹/L; hematocrit <45% (with or without phlebotomy in the previous 3 months or 12 weeks); absence of leukoerythroblastosis; absence of signs of progressive disease; absence of any hemorrhagic or thrombotic events; bone marrow histological remission (e.g., disappearance of megakaryocyte hyperplasia and absence of >grade 1 reticulin fibrosis; or presence of age-adjusted normocellularity and disappearance of trilinear hyperplasia, and absence of >grade 1 reticulin fibrosis); and molecular remission or response. A complete response (e.g., a complete hematological response) can be defined to include all or a subset of the criteria. A partial response (e.g., a partial hematological response) can be defined to include a smaller subset of the criteria.

For myelofibrosis (MF), e.g., associated with primary MF, post-polycythemia vera MF, and post-essential thrombocythemia MF, any combination of the following criteria can be used to define a response: age-adjusted normocellularity; <5% blasts; ≤grade 1 MF; hemoglobin ≥100 g/L and <UNL; neutrophil count ≥1×10⁹/L and <UNL; platelet count ≥100×10⁹/L and <UNL; <2% immature myeloid cells; resolution of disease symptoms; spleen and liver not palpable; no evidence of extramedullary hematopoiesis (EMH); hemoglobin ≥85 but <100 g/L and <UNL; platelet count ≥50, but <100×10⁹/L and <UNL; achievement of anemia, spleen or symptoms response without progressive disease or increase in severity of anemia, thrombocytopenia, or neutropenia; transfusion-independent patients: a ≥20 g/L increase in hemoglobin level; transfusion-dependent patients: becoming transfusion-independent; a baseline splenomegaly palpable at 5-10 cm, below the LCM, becomes not palpable; a baseline splenomegaly palpable at >10 cm, below the LCM, decreases by ≥50%; ≥35% spleen volume reduction; ≥50% reduction in the MPN-SAF TSS; cytogenic remission or response; and molecular remission or response.

A molecular response can include a reduction of one or more mutant allele burdens. For example, a molecular response can include a reduction in JAK2617F allele burden or in TET2 allele burden.

Allele burden (%) over time can be calculated. Allelic burden represents the percentage of mutant alleles present among all alleles of a particular gene in peripheral blood mononuclear cells. More specifically, the reduction of the allele burden can be at least 20%, 25%, 30%, 35%, 40%, 45%, 50%, 55%, 60%, 65%, 70%, 75%, 80%, 85%, 90%, 95%, or 99% or more, specifically between two time points or within a treatment period. The allele burden can decline to 50% or less, e.g., less than 50%, 45% or less, 40% or less, 37% or less, 35% or less, 30% or less, 25% or less, 20% or less, 15% or less, 10% or less, 7% or less, 5% or less, or 1% or less. A complete molecular response (CMR) is achieved when the allele burden is below the threshold of 1%. In some embodiments, the allele burden may be reduced to less than 0.01%.

Other indications of a good response can include a normal spleen size (measured via ultrasound measured via ultrasound; ≤12 cm for females, ≤13 cm for males), absence or low rate of any thromboembolic events, and a reduction of phlebotomy requirements by at least 50%, e.g., at least 55%, 60%, 65%, 70%, 75%, 80%, 85%. 90%, 95%, or 99%. A patient can be free of phlebotomy.

Complete hematologic response (CHR) can include hematocrit <45% (without phlebotomy in the previous 3 months), platelet count <400×10⁹/L, white blood cell count <10×10⁹ cells/L, with or without normal spleen size, and absence of thromboembolic events.

In some cases, the treated subject exhibits one or both of CHR and CMR during or at the end of a treatment period. The subject may be also free of phlebotomy and/or has a normal spleen size.

In any of the treatment methods described herein, the treatment responses (e.g., hematological response or molecular response) achieved by a subject having a TET2 mutant may not be significantly different from that achieved by a subject having a wild-type TET2. For example, the subject having a TET2 mutant may exhibit a similar level of response (e.g., level of or rate of reduction of JAK2617F allele burden) or timing of response (e.g., time to reach CHR).

In other words, the pegylated interferon-α described herein can be used to treat subjects regardless of their TET2 mutant status.

The specific example below is to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are hereby incorporated by reference in their entirety.

### EXAMPLE

PROUD-PV was a phase III, randomized, controlled, multicenter study conducted in 47 centers across Europe to compare ropeginterferon alpha-2b to hydroxyurea in patients with polycythemia vera. CONTINUATION-PV is the ongoing extension study of PROUD-PV.

Patients with polycythemia vera diagnosed using 2008 World Health Organization criteria who were either cytoreduction-naïve or hydroxyurea pre-treated without resistance or intolerance were enrolled. In PROUD-PV, patients were randomized 1:1 to receive ropeginterferon alpha-2b or hydroxyurea for one year. Thereafter, patients could roll over into CONTINUATION-PV. Treatment arms remained as allocated in PROUD-PV, but patients in the HU arm could switch to best-available therapy (BAT). Complete hematological response (CHR) was assessed according to modified European LeukemiaNet (ELN) criteria: (1) hematocrit <45% with no phlebotomy for ≥3 months; (2) platelet count <400×10⁹/L; (3) leukocyte count <10×10⁹/L.

Molecular response (MR) with respect to JAK2V617F allele burden was assessed according to European LeukemiaNet criteria.

Next generation sequencing (NGS; TruSight™ Myeloid Panel, Illumina) was performed to detect non-driver mutations. All patients enrolled in CONTINUATION-PV were included in an interim efficacy analysis over 36 months by non-driver baseline mutational status.

A total of 254 patients were treated in PROUD-PV and 171 rolled over into CONTINUATION-PV. All patients had a JAK2V617F mutation at screening. Baseline characteristics of the study cohort were balanced between the treatment arms and have been disclosed previously.

NGS data at baseline (at PROUD-PV enrolment) were available for 163/171 patients who entered CONTINUATION-PV. A TET2 mutation was identified in 14.1% of patients. Other frequently observed non-driver mutations affected DNMT3A (4.3%) and ASXL1 (1.2%).

Across both treatment arms, baseline characteristics (in PROUD-PV) showed that TET2mut patients were significantly older than TET2 wt patients (mean age: 65.3 years versus 56.1 years, respectively; p=0.0003), and a higher proportion of TET2mut patients were female (78.3% for TET2mut versus 47.1% for TET2 wt patients; p=0.007). See Table 1.

TABLE 1
Baseline characteristics by TET2 mutational status

| Variable | Statistics | TET2mut | | TET2wt | | p-value* |
|---|---|---|---|---|---|---|
| Age (years) | Mean (±SD) | 65.3 | (±9.49) | 56.1 | (±10.77) | 0.0003 |
| | Range | 45.0-85.0 | | 30.0-81.0 | | |
| Gender | Female, (n [%]) | 18 | (78.3%) | 66 | (47.1%) | 0.007 |
| | Male, (n [%]) | 5 | (21.7%) | 74 | (52.9%) | |
| Time since PV | Median (Q1-Q3) | 1.6 | (0.6-7.4) | 1.9 | (0.6-8.8) | 0.5 |
| diagnosis (months) | Range | 0.0-69.5 | | 0.0/145.5 | | |
| JAK2V617F allelic | Mean (±SD) | 51.2 | (±24.06) | 42.3 | (±22.50) | 0.09 |
| burden (%) | Range | 14.7-92.0 | | 5.0/94.9 | | |
| Hydroxyurea pre- | | N = 3 | | N = 46 | | |
| treated patients | | | | | | |
| Duration of pre-study | Median (Q1-Q3) | 9.4 | (1.5-23.1) | 9.5 | (2.7-25.1) | 0.8 |

| | | | |
|---|---|---|---|
| hydroxyurea treatment | Range | 1.5-23.1 | 0.9/36.4 |
| (months) | | | |

*Fishers exact test used for gender; for all other parameters the Willcoxon rank sum test was used.

Median JAK2V617F allele burden at baseline was higher in TET2mut patients than in TET2 wt patients (45.9% versus 37.3%, respectively), although the difference was not significant (p=0.09).

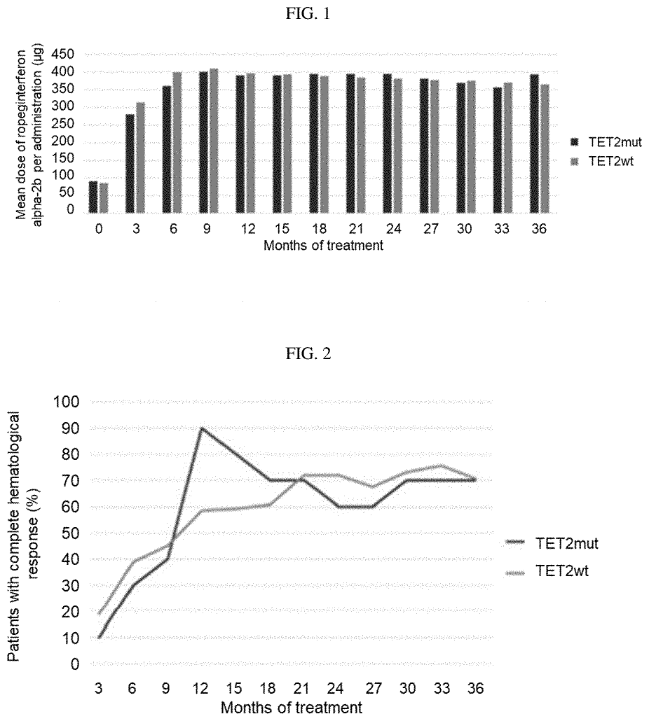

Dosing of ropeginterferon alpha-2b was comparable between the TET2mut and TET2 wt subgroups throughout treatment. See FIG. 1.

High hematological responses were achieved during long-term ropeginterferon alpha-2b therapy in both TET2mut and TET2 wt patients. At 24 and 36 months, CHR was achieved by 60.0% and 70.0% of TET2mut patients, respectively (n=10) and 72.0% and 70.7% of TET2 wt patients, respectively (n=82). See FIG. 2.

JAK2V617F molecular response rates to ropeginterferon alpha-2b showed no significant difference (P>0.05) between TET2mut and TET2 wt patients (50.0% at 24 and 36 months for TET2mut; 70.7% and 68.3%, respectively, for TET2 wt). Furthermore, there was no significant difference between the subgroups regarding the absolute JAK2V617F allele burden or the relative change in allele burden during treatment with ropeginterferon alpha-2b.

In TET2mut patients receiving ropeginterferon alpha-2b, the mean TET2 variant allele burden decreased steadily from 32.4% at baseline to 24.2%, 20.0% and 17.3% at months 12, 24 and 36, respectively, although only 3 TET2mut patients had available NGS data at month 36. The reduction was statistically significant at month 24 (p=0.047).

Mean JAK2V617F allele burden was reduced from 51.3% to 31.8% during ropeginterferon alpha-2b treatment in TET2mut patients.

The data demonstrated that ropeginterferon alpha-2b induced high hematological and molecular responses in patients with polycythemia vera regardless of TET2 mutational status.

Further, ropeginterferon significantly reduced the mutational burden of both JAK2 and TET2, thus reducing the mutated hematopoietic stem cell pool, underscoring the disease-modifying capability of this stem-cell targeting agent.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A method of reducing TET2 mutant allele burden in a subject, comprising:
administering to a subject identified as having a TET2 mutation a 50 to 540 μg dose of a pegylated interferon-α at a regular interval of 2 to 8 weeks for a treatment period, the pegylated interferon-α being a conjugate of formula I: 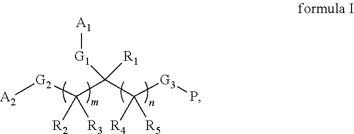
in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10,
wherein the subject has a reduction in TET2 mutant allele burden at a second time point in the treatment period as compared to a first time point before or earlier in the treatment period.

2. The method of claim 1, further comprising assaying and detecting the TET2 mutant allele burden in the subject at the first time point and the second time point.

3. The method of claim 1, further comprising assaying and detecting the TET2 mutant allele before the administering step.

4. The method of claim 2, wherein the second time point is 3 years or less after the first time point.

5. The method of claim 4, wherein the second time point is 1 years or less after the first time point.

6. The method of claim 1, where the reduction in TET2 mutant allele burden is maintained or increased beyond the second time point.

7. The method of claim 1, wherein the TET2 mutant allele burden is reduced by at least 10%.

8. The method of claim 7, wherein the TET2 mutant allele burden is reduced by at least 20%-50%.

9. The method of claim 1, wherein the subject has essential thrombocythemia, polycythemia vera or myelofibrosis.

10. The method of claim 1, wherein the subject has a complete hematologic response at the second time point.

11. The method of claim 1, where the subject has a reduced JAK2V617F allele burden at the second time point as compared to the first time point.

12. The method of claim 4, wherein the interval is 2 to 4 weeks.

13. The method of claim 1, the conjugate has one or more properties including:
(i) a median Tmax in the range of 3 to 6 days following administration of multiple 50 to 540 μg doses of the conjugate once every two weeks to subjects;
(ii) a mean T_{1/2} in the range of 6 to 10 days following administration of multiple 50 to 540 μg doses of the conjugate once every two weeks to subjects; and
(iii) an individual maximum tolerated dose of at least 500 μg once every 2 to 4 weeks in subjects.

14. The method of claim 1, wherein the conjugate has one or more features including: G3 is a bond and P is an interferon-α moiety in which the amino group at the N-terminus is attached to G3; A₁ and A₂ are polyalkylene oxide moieties having a molecular weight of 10-30 kD; each of G₁ and G₂ is 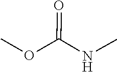
in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues.

15. The method of claim 1, wherein the interferon-α moiety is a human interferon-α_{2b} having an extra proline residue at the N-terminus and is 166 amino acids in length.

16. The method of claim 1, wherein the conjugate is 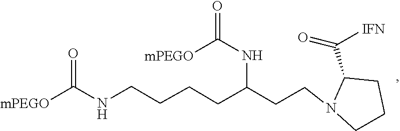
in which mPEG has a molecular weight of 20 kD and IFN is an interferon-α_{2b}.

17. A method of treating polycythemia vera in a subject, comprising:
administering to a subject identified as having polycythemia vera and a TET2 mutation a 50 to 540 μg dose of a pegylated interferon-α at a regular interval of 2-8 weeks for a treatment period, the pegylated interferon-α being a conjugate of formula I: 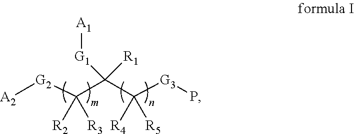
in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10,
wherein the subject has a reduction in JAK2V617F allele burden at a second time point in the treatment period as compared to a first time point before or earlier in the treatment period, and optionally, a complete hematological response also at the second time point.

18. The method of claim 17, further comprising assaying and detecting the TET2 mutant allele burden in the subject at the first time point and the second time point.

19. The method of claim 17, further comprising assaying and detecting the TET2 mutant allele before the administering step.

20. The method of claim 18, wherein the second time point is 3 years or less after the first time point.

21. The method of claim 18, wherein the second time point is 1 year or less after the first time point.

22. The method of claim 17, wherein the reduction in JAK2V617F allele burden is maintained or increased beyond the second time point.

23. The method of claim 17, wherein the complete hematological response is maintained beyond the second time point.

24. The method of claim 17, wherein the reduction in JAK2V617F allele burden is not significantly different from a control polycythemia vera subject with wild-type TET2.
